(19) United States
(12) Patent Application Publication
(10) Pub. No.: US 2006/0127506 A1
(43) Pub. Date: Jun. 15, 2006

(54) Compositions of S-adenosyl-L-methionine

(76) Inventor: Rolland F. Hebert, Seattle, WA (US)

Correspondence Address: ROLLAND HEBERT, 427 BELLEVUE AVE E. SUITE 301, SEATTLE, WA, 98102, US

(21) Appl. No.: 11/298,223

(22) Filed: Dec. 10, 2005

Related U.S. Application Data
(60) Provisional application No. 60/635,120, filed on Dec. 10, 2004.

(51) Int. Cl. A61K 36/38; A61K 31/714; A61K 31/7076; A61K 31/405; A61K 31/375; A61K 31/4415; A61K 31/455; A61K 31/525; A61K 31/51; A61K 31/355

(52) U.S. Cl. 424/730; 514/47; 514/251; 514/52; 514/276; 514/350; 514/356; 514/419; 514/458; 514/474

(57) ABSTRACT

Compositions of S-adenosyl-L-methionine with additives are disclosed.

### BACKGROUND CROSS-REFERENCES TO RELATED APPLICATION

This application claims the benefit of U.S. Provisional Patent Application Ser. No. 60/635,120 filed on Dec. 10, 2004.

### FIELD OF THE INVENTION

The present invention relates to novel compositions of matter containing substantially optically pure diastereomers of S-adenosyl-L-methionine and defined non-racemic ratios of (S,S)-S-adenosyl-L-methionine to (R,S)-S-adenosyl-L-methionine and other additives. These compositions possess potent activity in treating various conditions.

1. Technical Field

This patent relates to novel compositions of matter containing optically pure diastereomers of S-adenosyl-L-methionine (SAM-e), defined non-racemic ratios of (S,S)-S-adenosyl-L-methionine to (R,S)-S-adenosyl-L-methionine and certain additives and to therapeutic uses of these new compositions.

2. Background of the Invention

Many organic compounds exist in optically active forms, i.e., they have the ability to rotate the plane of plane-polarized light. In describing an optically active compound, the prefixes D and L or R and S are used to denote the absolute configuration of the molecule about its chiral center. The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are mirror images of one another. A specific stereoisomer may also be referred to as an enantiomer, and a mixture of such isomers is often called an enantiomeric mixture. A 50:50 mixture of enantiomers is referred to as a racemic mixture. A compound with more than one chiral center is a diastereomer. S-adenosyl-L-methionine is a diastereomer.

Stereochemical purity is of importance in the field of pharmaceuticals, where 12 of the 20 most prescribed drugs exhibit chirality. A case in point is provided by the L-form of the beta-adrenergic blocking agent, propranolol, which is known to be 100 times more potent than the D-enantiomer.

Furthermore, optical purity is important since certain isomers may actually be deleterious rather than simply inert. For example, it has been suggested that the D-enantiomer of thalidomide was a safe and effective sedative when prescribed for the control of morning sickness during pregnancy, and that the corresponding L-enantiomer was a potent teratogen.S-adenosyl-L-methionine is a naturally occurring substance that is present in all living organisms and has a number of very important biological functions. Among these functions are the following: methyl group donor in transmethylation reactions (it is the sole methyl group donor in such reactions-including methylation of DNA, proteins, hormones, catechol and indoleamines and phosphatidylethanolamine to phosphatidylcholine); it is a substrate of an enzyme lyase that converts S-adenosyl-L-methionine to the molecule methylthioadenosine and homoserine; it is an aminobutyric chain donor to tRNA; it is an aminoacidic chain donor in the biosynthesis of biotin; S-adenosyl-L-methionine, after decarboxylation, is the donor of aminopropyl groups for the biosynthesis of neuroregulatory polyamines spermidine and spermine. (Zappia et al (1979), Biomedical and Pharmacologcial roles of Adenosylmethionine and the Central Nervous System, page 1, Pergamon Press. NY.)

S-adenosyl-L-methionine has been used clinically in the treatment of liver disease (Friedel H, Goa, K. L., and Benfield P., (1989), S-adenosyl-L-methionine: a review of its pharmacological properties and therapeutic potential in liver dysfunction and affective disorders in relation to its physiological role in cell metabolism. Drugs. 38, 389-416), arthritis (Di Padova C, (1987), S-adenosyl-L-methionine in the treatment of osteoarthritis: review of the clinical studies. Am J. Med. 83, (Suppl. 5), 6-65), and depression (Kagan, B, Sultzer D. L., Rosenlicht N and Gerner R. (1990), Oral S-adenosyl-L-methionine in depression: a randomized, double blind, placebo-controlled trial. Am. J. Psychiatry 147, 591-595.) Alzheimer's patients have reduced cerebral spinal fluid levels of S-adenosyl-L-methionine (Bottiglieri et al, (1990), Cerebrospinal fluid S-adenosyl-L-methionine in depression and dementia: effects of treatment with parenteral and oral S-adenosyl-L-methionine. J. Neurol. Neurosurg. Psychiatry 53, 1096-1098.) In a preliminary study, S-adenosyl-L-methionine was able to produce cognitive improvement in patients with Alzheimer's disease. (Bottiglieri et al (1994), The clinical potential of admetionine (S-adenosyl-1-methioinine) in neurological disorders. Drugs 48, 137-152.) S-adenosyl-L-methionine brain levels in patients with Alzheimer's disease are also severely decreased. (Morrison et al, (1996), Brain S-adenosyl-L-methionine levels are severely decreased in Alzheimer's disease, Journal of Neurochemistry, 67, 1328-1331.) Patients with Parkinson's disease have also been shown to have significantly decreased blood levels of S-adenosyl-L-methionine. (Cheng et al, (1997), Levels of L-methionine S-adenosyltransferase activity in erythrocytes and concentrations of S-adenosyl-L-methionine and S-adenosylhomocysteine in whole blood of patients with Parkinson's disease. Experimental Neurology 145, 580-585.)

S-adenosyl-L-methionine levels in patients treated with the antineoplastic drug methotrexate are reduced. Neurotoxicity associated with this drug may be attenuated by co-administration of S-adenosyl-L-methionine. (Bottiglieri et al (1994), The Clinical Potential of Ademetionine (S-adenosyl-L-methionine) in neurological disorders, Drugs, 48 (2), 137-152.) Cerebral spinal fluid levels of S-adenosyl-L-methionine have been investigated in HIV AIDS dementia Complex/HIV encephalopathy and found to be significantly lower than in non-HIV infected patients. (Keating et al (1991), Evidence of brain methyltransferase inhibition and early brain involvement in HIV positive patients Lancet: 337:935-9.)

De La Cruz et al have shown that S-adenosyl-L-methionine, chronically administered, can modify the oxidative status in the brain by enhancing anti-oxidative defenses. (De La Cruz et al, (2000), Effects of chronic administration of S-adenosyl-L-methionine on brain oxidative stress in rats. Naunyn-Schmiedeberg's Archives Pharmacol 361: 47-52.) This is similar to results obtained with S-adenosyl-L-methionine in liver and kidney tissue. Thus S-adenosyl-L-methionine would be useful as an antioxidant. Oral S-adenosyl-L-methionine administration to patients with and without liver disease has resulted in increases in liver glutathione levels. (Vendemiale G et al, (1989), Effect of oral S-adenosyl-L-methionine on hepatic glutathione in patients with liver disease. Scand J Gastroenterol; 24: 407-15. Oral administration of S-adenosyl-L-methionine to patients suffering from intrahepatic cholestasis had improvements in both the pruritus as well as the biochemical markers of cholestasis. (Giudici et al, The use of admethionine (S-adenosyl-L-methionine) in the treatment of cholestatic liver disorders. Meta-analysis of clinical trials. In: Mato et al editors. Methionine Metabolism: Molecular Mechanism and Clinical Implications. Madrid: CSIC Press; 1992 pp 67-79.) Oral S-adenosyl-L-methionine administration to patients suffering from primary fibromyalgia resulted in significant improvement after a short term trial. (Tavoni et al, Evaluation of S-adenosylmethioine in Primary Fibromaylgia. The American Journal of Medicine, Vol 83 (suppl 5A), pp 107-110, 1987.) S-adenosyl-L-methionine has been used for the treatment of osteoarthritis as well. (Koenig B. A long-term (two years) clinical trial with S-adenosyl-L-methionine for the treatment of osteoarthritis. The American Journal of Medicine, Vol 83 (suppl 5A), Nov. 20, 1987 pp 89-94)

S-adenosyl-L-methionine is clinically useful in many apparently unrelated areas because of its important function in basic metabolic processes. One of its most striking clinical uses is in the treatment of alcoholic liver cirrhosis that, until now, remained medically untreatable. Mato et al demonstrated the ability of oral S-adenosyl-L-methionine in alcoholic liver cirrhosis to decrease the overall mortality and/or progression to liver transplant by 29% vs 12% as compared with a placebo treated group. (Mato et al (1999), S-adenosyl-L-methionine in alcohol liver cirrhosis: a randomized, placebo-controlled, double blind, multi-center clinical trial, Journal of Hepatology, 30, 1081-1089.)

S-adenosyl-L-methionine also attenuates the damage caused by tumor necrosis factor alpha and can also decrease the amount of tumor necrosis factor alpha secreted by cells. Consequently, conditions in which this particular inflammatory factor is elevated would benefit from the administration of S-adenosyl-L-methionine. (Watson W H, Zhao Y, Chawla R K, (1999) Biochem J Aug 15; 342 (Pt 1):21-5. S-adenosyl-L-methionine attenuates the lipopolysaccharide-induced expression of the gene for tumour necrosis factor alpha) S-adenosyl-L-methionine has also been studied for its ability to reduce the toxicity associated with administration of cyclosporine A, a powerful immunosuppressor. (Galan A, et al, Cyclosporine. A toxicity and effect of the S-adenosyl-L-methionine, Ars Pharmaceutica, 40:3; 151-163, 1999.)

S-adenosyl-L-methionine, incubated in vitro with human erythrocytes, penetrates the cell membrane and increases ATP within the cell thus restoring the cell shape. (Friedel et al, S-adenosyl-L-methionine: A review of its pharmacological properties and therapeutic potential in liver dysfunction and affective disorders in relation to its physiological role in cell metabolism, Drugs 38 (3):389-416, 1989)

S-adenosyl-L-methionine has been studied in patients suffering from migraines and found to be of benefit. (Friedel et al, S-adenosyl-L-methionine: A review of its pharmacological properties and therapeutic potential in liver dysfunction and affective disorders in relation to its physiological role in cell metabolism, Drugs 38 (3): 389-416, 1989)

Belli et al in an article entitled “S-adenosylmethionine prevents total parenteral nutrition-induced cholestasis in the rat”, Journal of Hepatology 1994; 21: 18-23 showed that S-adenosyl-L-methionine was able to prevent cholestasis resulting from total parenteral nutrition by maintaining liver plasma membrane enzymatic activity via preservation of the membrane lipid environment.

S-adenosyl-L-methionine has been administered to patients with peripheral occlusive arterial disease and was shown to reduce blood viscosity, chiefly via its effect on erythrocyte deformability.

Garcia P et al in “S-adenosylmetionine: A drug for the brain?”, IV th Workshop on Methionine Metabolism: Molecular Mechanisms and Clinical Implications”, Symposium held on March 1-5, Granada, Spain, 1998 reported that S-adenosyl-L-methionine was able to increase the number of muscarinic receptors in the brains of rats treated chronically with S-adenosyl-L-methionine. Muscarinic receptors in the brain, especially in the hippocampus, are important in learning and memory. In a standard eight arm radical maze test, treated animals were able to out-perform age matched older untreated animals. Young aged matched S-adenosyl-L-methionine treated animals were also able to out-perform young non treated animals showing S-adenosyl-L-methionine's ability to increase memory even in young animals. The conclusions drawn were that S-adenosyl-L-methionine is able to improve memory not only in adult aged animals but also in young animals thus making S-adenosyl-L-methionine an eligible candidate therapy for the treatment of memory impairment and learning difficulties.

S-adenosyl-L-methionine is commercially available using fermentation technologies that result in S-adenosyl-L-methionine formulations varying between 60 and 80% purity. (That is, the final product contains 60-80% of the active or (S,S)-S-adenosyl-L-methionine and 20-40% of the inactive or (R,S)-S-adenosyl-L-methionine.) (Gross, A., Geresh, S., and Whitesides, Gm (1983) Appl. Biochem. Biotech. 8, 415.) Enzymatic synthetic methodologies have been reported to yield the inactive isomer in concentrations exceeding 60%. (Matos, J R, Rauschel F M, Wong, C H. S-adenosyl-L-methionine: Studies on Chemical and Enzymatic Synthesis. Biotechnology and Applied Biochemistry 9, 39-52 (1987). Enantiomeric separation technologies have been reported to resolve the pure active diastereomer of S-adenosyl-L-methionine. (Matos, J R, Rauschel F M, Wong, C H. S-adenosyl-L-methionine: Studies on Chemical and Enzymatic Synthesis. Biotechnology and Applied Biochemistry 9, 39-52 (1987; Hoffman, Chromatographic Analysis of the Chiral and Covalent Instability of S-adenosyl-L-methionine, Biochemistry 1986, 25 4444-4449: Segal D and Eichler D, The Specificity of Interaction between S-adenosyl-L-methionine and a nucleolar 2-O-methyltransferase, Archives of Biochemistry and Biophysics, Vol. 275, No. 2, December, pp. 334-343, 1989) Newer separation technologies exist to resolve enantiomers and diastereomers on a large commercial production scale at a very economic cost.

It is well known in the art that S-adenosyl-L-methionine is highly unstable during the manufacturing process irrespective of the manner in which it is made, that is, yeast or bacterial fermentation or synthetic methodologies. Matos and Wong, in Bioorganic Chemistry 15, 71-80, 1987 show that the epimeric instability of the S-adenosyl-L-methionine molecule in solution is a temperature dependent phenomenon despite the counter ion used to stabilize the molecule. During the fermentation process to obtain the S-adenosyl-L-methionine (whether bacterial or yeast) the temperature of the solutions tends to be elevated thus compromising the diastereomeric stability of the S-adenosyl-L-methionine molecule during the process. In United States Patent Application 20020010147 Berna, Marco; et al. Jan. 24, 2002 disclose a process for the manufacture of high percentage of (S,S)S-adenosyl-L-methionine:(R,S)S-adenosyl-L-methionine. They disclose an optical purity of 97-100% (S,S)S-adenosyl-L-methionine:(R,S)S-adenosyl-L-methionine. However, it is noteworthy that this patent does not disclose a methodology for the use of these higher percentages of optically pure (S,S)S-adenosyl-L-methionine:(R,S)S-adenosyl-L-methionine salts with other additives such as those that are the object of this present invention. In addition it is noteworthy to mention that this optically purity as disclosed in the patent application is not sustainable beyond 4 months when the epimerization of the molecule begins again.

The present inventor has over a number of years of striving to stabilize (S,S) S-adenosyl-L-methionine noted that it is necessary to have as high as a concentration as possible of (S,S) S-adenosyl-L-methionine in the final formulation of the drug and this will ultimately slow down the epimerization process of the (S,S)S-adenosyl-L-methionine to (R,S)S-adenosyl-L-methionine but will not halt it altogether.

The present inventor has, in a previous U.S. patent application Ser. No. 11/136,271 filed on May 24, 2005, disclosed methodologies to produce the highest possible concentrations of (S,S)S-adenosyl-L-methionine. This patent pending application Ser. No. 11,/136271 is incorporated its entirety herein and discloses not only new salts of S-adenosyl-L-methionine but also methods for the production of the optically pure or defined non-racemic mixture of the two diastereomers of (S,S)S-adenosyl-L-methionine:(R,S)S-adenosyl-L-methionine salts used in this present patent application and to which can be added the compounds disclosed.

De la Haba first showed that the sulfur is chiral and that only one of the two possible configurations was synthesized and used biologically. (De la Haba et al J. Am. Chem. Soc. 81, 3975-3980, 1959) Methylation of RNA and DNA is essential for normal cellular growth. This methylation is carried out using S-adenosyl-L-methionine as the sole or major methyl donor with the reaction being carried out by a methyltransferase enzyme. Segal and Eichler showed that the enzyme bound (S,S)-S-adenosyl-L-methionine 10 fold more tightly than (R,S)-S-adenosyl-L-methionine thus demonstrating a novel binding stereospecificity at the sulfur chiral center. Other methyltransferases have been reported to bind (R,S)-S-adenosyl-L-methionine to the same extent as (S,S)-S-adenosyl-L-methionine and thus (R,S)-S-adenosyl-L-methionine could act as a competitive inhibitor of that enzyme. (Segal D and Eichler D, The Specificity of Interaction between S-adenosyl-L-methionine and a nucleolar 2-O-methyltransferase, Archives of Biochemistry and Biophysics, Vol. 275, No. 2, December, pp. 334-343, 1989; Borchardt R T and Wu Y S, Potential inhibitors of S-adenosyl-L-methionine-dependent methyltransferases. Role of the Asymmetric Sulfonium Pole in the Enzymatic binding of S-adenosyl-L-methionine, Journal of Medicinal Chemistry, 1976, Vol 19, No. 9, 1099-1103.)

Borchardt and Wu, in an article entitled “Potential Inhibitors of S-adenosyl-L-methionine-dependent methyltransferases. 5. Role of the Asymmetric Sulfonium Pole in the Enzymatic Binding of Adenosyl-L-methionine”, Journal of Medicinal Chemistry, 1976, Vol. 19, No. 9, pp 1099-1103, report that the (+)-SAM (no longer used nomenclature for (R,S)-S-adenosyl-L-methionine) is a potent inhibitor of enzyme-catalyzed transmethylation reactions. Since transulferation and methylation reactions are the hallmark of S-adenosyl-L-methionine's mechanism of action, it would be prudent to use S-adenosyl-L-methionine with as enriched a concentration of (S,S)-S-adenosyl-L-methionine in any pharmaceutical composition as possible since the. (R,S)-S-adenosyl-L-methionine diastereomer may be inhibitory to the desired action of (S,S)-S-adenosyl-L-methionine. Alternately and interestingly, the (R,S)-S-adenosyl-L-methionine diastereomer might conceivably be used as a novel anticancer agent since it may be able to inhibit DNA methyltransferase activity. (Karpf et al “Inhibition of DNA methyltransferase stimulates the expression of signal transducer and activator of transcription 1,2, and 3 genes in colon tumor cells. PNAS Nov. 23, 1999, vol 96, no. 24, 14007-14012. Consequently it would be even more prudent to separate this diastereomer from the (S,S)-S-adenosyl-L-methionine diastereomer in order to completely separate the opposite activities that these two molecules possess. (Detich et al in an article entitled “The methyl donor S-adenosyl-L-methionine inhibits active demethylation of DNA; a candidate novel mechanism for the pharmacological effects of S-adenosylmethionine.” J Biol Chem. 2003 Jun. 6;278(23):20812-20, point out the tumor protective mechanism of S-adenosyl-L-methionine and the importance of intracellular S-adenosyl-L-methionine concentrations in cancer prevention. Presumably this is due to the ability of S-adenosyl-L-methionine to prevent DNA hypomethylation. Indeed, DNA hypomethylation is a hallmark of cancer cells and the correction of this hypomethylation leads to proper gene expression and reversal or prevention of cancer. However, in light of the known inability of (R,S)-S-adenosyl-L-methionine to participate in methylation or transulfuration reactions (indeed, it inhibits these reactions), it becomes increasingly apparent that S-adenosyl-L-methionine compositions should contain the least amount of (R,S)-S-adenosyl-L-methionine possible.

It is further known that certain additives both of natural as well of synthetic origin may either enhance the effects of S-adenosyl-L-methionine or make its administration easier. Thus, the compositions of this current patent application will enhance the effect of S-adenosyl-L-methionine and make its administration much easier.

### PRIOR ART

In U.S. Pat. No. 6,649,753 issued Nov. 18,m 2003, Deshpande et al disclose stable salts of using resorcinol-4-6-disulfonic acid, catechol-3,5-disulfonic acid and phenol-2,4,6-trisulfonic acid (S-adenosyl-L-methionine) and the process for their preparation. However, while the authors disclose stable salts of S-adenosyl-L-methionine with enrichment of (S,S)S-adenosyl-L-methionine to 65% using resorcinol-4-6-disulfonic acid, catechol-3,5-disulfonic acid and phenol-2,4,6-trisulfonic acid to make the stable salts, they do not disclose the use of resorcinol-4-6-disulfonic acid, catechol-3,5-disulfonic acid and phenol-2,4,6-trisulfonic acid to stabilize more purified (S,S)S-adenosyl-L-methionine salt to from 65.001%-100% of the diastereomer. In addition, they do not disclose the use of a stabilized (R,S)S-adenosyl-L-methionine salt from about 34.009%-100%. Additionally, they do not point out a defined non-racemic mixture of the two diastereomers of (S,S)S-adenosyl-L-methionine/(R,S)S-adenosyl-L-methionine salts. They also do not disclose the use of the additives that are the subject of the present invention.

In patent PCT application WO 02/083703 A1, Derrieu et al disclose new stable salts of S-adenosyl-L-methionine with polyphosphates but do not disclose the use of the purified diastereomers of S-adenosyl-L-methionine or a defined non-racemic mixture of the two diastereomers of (S,S)S-adenosyl-L-methionine/(R,S)S-adenosyl-L-methionine salts made with polyphosphates. They also do not disclose the use of the additives of the present invention.

In United States Patent Application 20020010147 Berna, Marco; et. al. Jan. 24, 2002 disclose a process for the manufacture of high percentage of (S,S)S-adenosyl-L-methionine: (R,S)S-adenosyl-L-methionine. They disclose an optical purity of 97-100% (S,S)S-adenosyl-L-methionine:(R,S) S-adenosyl-L-methionine. However, Berna et al do not disclose the use of the additives of the present invention nor do they disclose the use of those additives with (S,S)S-adenosyl-L-methionine: (R,S)S-adenosyl-L-methionine ratios less than 97% concentration for (S,S)S-adenosyl-L-methionine or greater than 3% for (R,S)S-adenosyl-L-methionine diastereomer.

The present inventor, in previously issued or pending patent applications, (U.S. Pat. No. 6,635,615, Hebert Oct. 21, 2003, Stable salts of S-adenosyl-L-methionine and United States Patent Application 20020025926, Hebert, Rolland F.

Feb. 28, 2002, Ehantiomers of S-adenosyl-L-methionine and United States Patent Application, 20030144244, Hebert, Rolland F., Jul. 31, 2003, Stable compositions of S-adenosyl-L-methionine with dextran these are all incorporated by reference in their entirety), disclosed stable salts of S-adenosyl-L-methionine as well as substantially optically pure (S,S)-sadenosylmethionine or a defined non-racemic ratio of (S,S)S-adenosyl-L-methionine to (R,S)S-adenosyl-L-methionine and methods for their use.

The diastereomers of S-adenosyl-L-methionine may be administered in combination with other natural or synthetic substances in a single pill or solution to make patient compliance much better since it is well known in clinical science that the fewer number of medications a patient must take increases the chances that the patient will comply with the medical orders.

Administration of the new compositions of optically pure diastereomers of S-adenosyl-L-methionine or defined non-racemic ratios of (S,S)-S-adenosyl-L-methionine to (R,S)-S-adenosyl-L-methionine and their salts (any salt of S-adenosyl-L-methionine that has been previously disclosed in the patent or scientific literature is contemplated in this present patent application) along with the additives as disclosed in the present invention would have significant utility over a wide range of disorders or conditions associated with low levels of S-adenosyl-L-methionine or conditions of DNA or RNA hypomethylation. Since the two diastereomeric forms of S-adenosyl-L-methionine of the present invention do not exhibit the same biological activity but rather that the (R,S)S-adenosyl-L-methionine diastereomer exhibits competitive inhibition, it is necessary for a rational pharmaceutical therapy to use the more active diastereomeric form of S-adenosyl-L-methionine. In this regard, and in view of the fact that (R,S)-S-adenosyl-L-methionine diastereomer acts as a competitive inhibitor of (S,S,) S-adenosyl-L-methionine in methyltransferase reactions, a more ideal S-adenosyl-L-methionine composition would be the substantially optically pure biologically active (S, S)—S-adenosyl-L-methionine form or a defined non-racemic ratio of (S,S)-S-adenosyl-L-methionine to (R,S)-S-adenosyl-L-methionine to include the highest possible concentration of the (S,S)-S-adenosyl-L-methionine form.

It is an object of the present invention to provide compositions of S-adenosyl-L-methionine containing substantially pure biologically active (S, S)S-adenosyl-L-methionine or a defined non-racemic ratio of (S,S)-S-adenosyl-L-methionine to (R,S)-S-adenosyl-L-methionine and additives. It is a further object of the present invention to provide methods of treatment or prevention of conditions in warm blooded animals that are related to lowered S-adenosyl-L-methionine levels, or hypomethylatio of RNA or DNA, by administering the compositions of the present invention.

In addition, it is known that S-adenosyl-L-methionine is unstable in terms of its diastereomers. That is, S-adenosyl-L-methionine epimerizes over time in powder form as well as in solution from (S,S)S-adenosyl-L-methionine the biologically active diastereomer to (R,S)S-adenosyl-L-methionine the form that inhibits the very methylation reaction for which S-adenosyl-L-methionine is needed. Therefore, it is one more object of the present invention to provide relatively stable compositions of S-adenosyl-L-methionine with the additives that are disclosed in this present invention.

Accordingly, there is need in the art for compositions and methods related to the use of substantially optically pure diastereomeric forms of S-adenosyl-L-methionine and defined non-racemic ratios of (S,S)-S-adenosyl-L-methionine to (R,S)-S-adenosyl-L-methionine along with additives of the present invention to increase blood and other tissue and fluid levels of S-adenosyl-L-methionine and to treat and prevent conditions which result from low blood and tissue levels of S-adenosyl-L-methionine. The author of this present invention fulfills these needs, and provides further related advantages.

### SUMMARY OF THE INVENTION

Briefly stated, the present invention discloses compositions of substantially optically pure diastereomeric forms of S-adenosyl-L-methionine, defined non-racemic ratios of (S,S)-S-adenosyl-L-methionine to (RS)-S-adenosyl-L-methionine and, their salts with certain additives to improve the efficacy of the S-adenosyl-L-methionine or to render its administration to mammals in need thereof easier. The compositions of this present invention have utility in increasing blood, RNA methylation and DNA methylation levels and other tissue or fluid levels of S-adenosyl-L-methionine, as well as treating or preventing a wide variety of conditions in warm blooded animals associated with low RNA methylation, DNA methylation, protein methylation, blood or other tissue or fluid levels of S-adenosyl-L-methionine. In addition, this present invention discloses methods of use of these compositions in the treatment and the prevention of diseases associated with low tissue and blood levels of S-adenosyl-L-methionine in mammals.

Thus in one embodiment, a composition of a substantially optically pure diastereomeric form of S-adenosyl-L-methionine salt or defined non-racemic ratios of (S,S)-S-adenosyl-L-methionine to (R,S)-S-adenosyl-L-methionine and their salts along with the additives of the present invention is administered to a warm-blooded animal in need thereof to increase tissue, cellular, RNA methylation, DNA methylation, protein methylation and blood S-adenosyl-L-methionine levels. In another embodiment, a composition of a substantially optically pure diastereomeric form of S-adenosyl-L-methionine salt or defined non-racemic ratios of (S,S)—S-adenosyl-L-methionine to (R,S)-S-adenosyl-L-methionine and their salts along with additives of this present invention is administered to a warm-blooded animal in need thereof to prevent or treat a condition associated with low cellular, RNA methylation, DNA methylation, protein methylation, and tissue and blood levels of S-adenosyl-L-methionine.

In yet a further embodiment, a composition of a substantially optically pure diastereomeric form of S-adenosyl-L-methionine salt or defined non-racemic ratios of (S,S)-S-adenosyl-L-methionine to (R,S)-S-adenosyl-L-methionine and their salts along with additives of the present invention is administered to a warm blooded animal to prevent and or treat the following conditions: aging, aging of the skin, Alzheimer's disease, rheumatoid arthritis, osteoarthritis, both as an anti-inflammatory as well as to promote new cartilage formation, cancer, conditions of hypomethylation, mitochondrial diseases, hypomethylation of DNA and RNA, nerve damage associated with HIV/AIDS, anxiety, attention deficit disorder and ADHD, sleep regulation, organ preservation for transplant industry, dyslipidemias, excess sebum production, migraines, bile dysfunction caused by pregnancy and use of contraceptive medications, depression, acute and chronic liver disease, cirrhosis of the liver, ischemic reperfusion injury, Parkinson's disease, memory disturbances, memory loss, pancreatitis, intrahepatic cholestasis, inflammation, pain, side effects of administration of chemotherapy, liver disease associated with administration of total parenteral nutrition, liver dysfunction, low tissue levels of glutathione, administration of neuroleptic drugs, administration of cyclosporin A, asthma, diabetes and alcohol withdrawal.

### DETAILED DESCRIPTION OF THE INVENTION

As mentioned above, this invention is generally directed to compositions of a substantially optically pure diastereomeric form of S-adenosyl-L-methionine salts and to defined non-racemic ratios of (S,S)-S-adenosyl-L-methionine to (R,S)-S-adenosyl-L-methionine and their salts along with additives that either increase the efficacy of S-adenosyl-L-methionine or render its administration easier. Such a composition of a substantially optically pure diastereomeric form of S-adenosyl-L-methionine salt or defined non-racemic ratios of (S,S)—S-adenosyl-L-methionine to (R,S)-S-adenosyl-L-methionine and their salts along with additives of the present invention is administered to a warm-blooded animal in need thereof to prevent or treat a condition associated with low cellular, tissue and blood levels of S-adenosyl-L-methionine.

As used herein, the term “conditions” includes diseases, injuries, disorders, indications and/or afflictions that are associated with decreased levels of blood and tissue S-adenosyl-L-methionine as well as decreased RNA methylation, DNA methylation, protein methylation The term “treat” or “treatment” means that the symptoms associated with one or more conditions associated with low levels of S-adenosyl-L-methionine or DNA, RNA or protein s hypomethylation are alleviated or reduced in severity or frequency and the term “prevent” means that subsequent occurrences of such symptoms are avoided or that the frequency between such occurrences is prolonged.

The term “substantially optically pure as used herein, means that the composition contains greater than about 81. % of the (S,S)-S-adenosyl-L-methionine diastereomer by weight in relation to the (R,S) diastereomer of S-adenosyl-L-methionine, preferably greater than about 94% of the (S,S)-S-adenosyl-L-methionine by weight, and more preferably greater than about 96.00% of (S,S)-S-adenosyl-L-methionine by weight, based upon the total weight of S-adenosyl-L-methionine.

The substantially optically pure diastereomeric forms of S-adenosyl-L-methionine salts or defined non-racemic ratios of (S,S)-S-adenosyl-L-methionine to (R,S)-S-adenosyl-L-methionine and their salts as well as the additives envisioned in this patent application may be used to prevent and/or treat a variety of conditions associated with lowered levels of S-adenosyl-L-methionine. Due to its ubiquitous distribution in mammalian tissue, S-adenosyl-L-methionine is associated with a variety of conditions: aging, aging of the skin, Alzheimer's disease, rheumatoid arthritis, osteoarthritis, both as an anti-inflammatory as well as to promote new cartilage formation, cancer, conditions of hypomethylation, mitochondrial diseases, hypomethylation of DNA and RNA and proteins, HIV/AIDS, anxiety, attention deficit disorder and ADHD, sleep regulation, organ preservation for transplant industry, dyslipidemias, excess sebuin production, migraines, bile dysfunction caused by pregnancy and use of contraceptive medications, depression, acute and chronic liver disease, cirrhosis of the liver, ischemic reperfusion injury, Parkinson's disease, memory disturbances, memory loss, pancreatitis, intrahepatic cholestasis, inflammation, pain, side effects of administration of chemotherapy, liver disease associated with administration of total parenteral nutrition, liver dysfunction, low tissue levels of glutathione, administration of neuroleptic drugs, administration of cyclosporin A, asthma, and alcohol-withdrawal.

Accordingly, compositions of optically pure diastereomers of S-adenosyl-L-methionine or defined non-racemic ratios of (S,S)-S-adenosyl-L-methionine to (R,S)-S-adenosyl-L-methionine and their salts along with the additives of the present invention are effective in preventing and/or treating the above conditions due to their ability to increase S-adenosyl-L-methionine levels or to increase RNA methylation, DNA methylation, or protein methylation To this end, compositions of optically pure diastereomers of S-adenosyl-L-methionine or defined non-racemic ratios of (S,S)-S-adenosyl-L-methionine to (R,S)-S-adenosyl-L-methionine and their salts along with the additives of the present invention may be used for pharmaceutical, prophylactic and/or cosmetic purposes, and are administered to a warm-blooded animal in an effective amount to achieve a desired result.

In the case of pharmaceutical administration, an effective amount of the composition is a quantity sufficient to treat the symptoms of a condition and/or the underlying condition itself. An effective amount of the composition in the context of prophylactic administration means an amount sufficient to avoid or delay the onset of a condition and/or its symptoms. Lastly, an effective amount with regard to cosmetic administration is an amount sufficient to achieve the desired cosmetic result.

In the current medical context, an effective amount of a substantially optically pure diastereomeric form of S-adenosyl-L-methionine salts or a non-racemic mixture of (S,S)-S-adenosyl-L-methionine and (R,S)-S-adenosyl-L-methionine and their salts would be in the range of from 200 mg per day to 5 grams per day or greater depending upon the specific condition of the patient to be treated or prevented. Thus, it is possible that doses higher than 5 grams per day may be needed in the event of the treatment of cancer but less than 400 mg per day in the treatment or prevention of minor aches and pains associated with mild arthritis. However, it is well known in the medical art how one arrives at the appropriate dosage. In addition, it is well known in the art how one arrives at the determination of both blood and tissue levels of S-adenosyl-L-methionine as well as any of the additives envisioned by this present patent application since NMR, HPLC as well as capillary electrophoresis methods are well known in the art to determine S-adenosyl-L-methionine.

### EMBODIMENTS

It is well known in medicine that patient compliance is an important issue when treating or preventing disease. It is therefore an object of the present invention to provide for compositions and methods to treat and prevent a disease that responds to S-adenosyl-L-methionine by incorporating one or more important additives along with the S-adenosyl-L-methionine in the same capsule, liquid, tablet, IV or 1M solution, or pill form that the patient may then take without having to take multiple capsules, liquids, tablets or pills. Thus for each of the different indications for which S-adenosyl-L-methionine is known, this present invention envisions an appropriate additive to be combined with substantially optically pure diastereomeric form of S-adenosyl-L-methionine salts or a non-racemic mixture of (S,S)-S-adenosyl-L-methionine and (R,S)-S-adenosyl-L-methionine and their salts.

For the treatment or prevention of liver disease associated with a viral causation, for example, substantially optically pure diastereomeric form of S-adenosyl-L-methionine salts or a non-racemic mixture of (S,S)-S-adenosyl-L-methionine and (R,S)-S-adenosyl-L-methionine and their salts in any of the forms envisioned by this present invention may be combined with an appropriate antiviral medication such as ribavirin and/or an immune modulation medication such as interferon or TNF alpha inhibitor in any of their pharmaceutically acceptable forms such as in a capsule, pill, liquid, injection, tablet or any other pharmaceutically acceptable manner and taken in the doses and manner prescribed and appropriate for the patient's condition. In addition, the substantially optically pure diastereomeric form of S-adenosyl-L-methionine salts or a non-racemic mixture of (S,S)-S-adenosyl-L-methionine and (R,S)-S-adenosyl-L-methionine and their salts can also be combined with the following including but not limited to folic acid at 50 to 1000 mcg; vitamin B6 at 10 to 2000 mg; vitamin B12 at 10-3000 mcg and biopterin, its precursors, or derivatives at 25-2000 mg; and vitamin C at 30-500 mg.

For the treatment or prevention of liver disease caused by excessive alcohol consumption, substantially optically pure diastereomeric form of S-adenosyl-L-methionine salts or a non-racemic mixture of (S,S)-S-adenosyl-L-methionine and (R,S)-S-adenosyl-L-methionine and their salts as envisioned by the present invention, may be combined with the following including but not limited to folic acid at 50 to 1000 mcg; vitamin B6 at 10 to 2000 mg; vitamin B12 at 10-3000 mcg and biopterin or any other vitamins deemed necessary to either enhance the effect of the substantially optically pure diastereomeric form of S-adenosyl-L-methionine salts or a non-racemic mixture of (S,S)-S-adenosyl-L-methionine and (R,S)-S-adenosyl-L-methionine and their salts in this particular patient population or to effectively treat an underlying nutritional deficiency thus providing an easier way to take the medications rather than to require the patient to take each medication separately. The doses of the additives to be administered in combination with the substantially optically pure diastereomeric form of S-adenosyl-L-methionine salts or a non-racemic mixture of (S,S)-S-adenosyl-L-methionine and (R,S)-S-adenosyl-L-methionine and their salts are well known and can be adjusted according to the medical need of the particular patient population.

For the treatment or prevention of arthritis, substantially optically pure diastereomeric forms of S-adenosyl-L-methionine salts or a non-racemic mixture of (S,S)-S-adenosyl-L-methionine and (RS)-S-adenosyl-L-methionine and their salts as envisioned by the present invention, may be combined with, for example, glucosamine (at doses ranging from 500 mg to 2 grams per day), chondrotin sulfate, or any known COX2 inhibitors (given at currently well known daily doses to achieve symptom relief) or any other TNF alpha inhibitor or any non-steriodal anti-inflammatory drugs to improve, or hasten the anti-inflammatory and analgesic effect of the substantially optically pure diastereomeric form of S-adenosyl-L-methionine salts or a non-racemic mixture of (S,S)-S-adenosyl-L-methionine and (R,S)-S-adenosyl-L-methionine and their salts and with the following additives including but not limited to folic acid at 50 to 1000 mcg; vitamin B6 at 10 to 2000 mg; vitamin B12 at 10-3000 mcg and biopterin.

In addition, substantially optically pure diastereomeric forms of S-adenosyl-L-methionine salts or a non-racemic mixture of (S,S)-S-adenosyl-L-methionine and (RS)-S-adenosyl-L-methionine and their salts may be combined with any immune system modulating drugs to treat or prevent arthritis due to autoimmune diseases such as rheumatoid arthritis. Thus, for example, substantially optically pure diastereomeric forms of S-adenosyl-L-methionine salts or a non-racemic mixture of (S,S)-S-adenosyl-L-methionine and (R,S)-S-adenosyl-L-methionine and their salts of this present patent application might be combined with current standard anti-rheumatoid arthritis treatment.

For the treatment or prevention of memory loss, substantially optically pure diastereomeric forms of S-adenosyl-L-methionine salts or a non-racemic mixture of (S,S)-S-adenosyl-L-methionine and (R,S)-S-adenosyl-L-methionine and their salts as envisioned by the present invention may be combined with botanical medicines such as gingko biloba that have been shown to be helpful in memory loss, or with conventional pharmaceutical medicines such as acetylcholinesterase inhibitors, gama secretase inhibitors, or gaba inhibitors (given at currently well known daily doses) and including but not limited to folic acid at 50 to 1000 mcg; vitamin B6 at 10 to 2000 mg; vitamin B12 at 10-3000 mcg and biopterin In addition, substantially optically pure diastereomeric forms of S-adenosyl-L-methionine salts or a non-racemic mixture of (S,S)-S-adenosyl-L-methionine and (R,S)-S-adenosyl-L-methionine and their salts as envisioned by this present invention may be combined with any nutritional supplements that may be helpful in the treatment or prevention of memory loss such as for example, vitamin E, lipoic acid.

For the treatment or prevention of Alzheimer's disease, for example, substantially optically pure diastereomeric forms of S-adenosyl-L-methionine salts or a non-racemic mixture of (S,S)-S-adenosyl-L-methionine and (R,S)-S-adenosyl-L-methionine and their salts as envisioned by the present invention may be combined with conventional pharmaceutical medicines such as acetylcholinesterase inhibitors to provide a synergistic effect in treatment or prevention of the disease and including but not limited to folic acid at 50 to 1000 mcg; vitamin B6 at 10 to 2000 mg; vitamin B12 at 10-3000 mcg and biopterin.

In addition, for the treatment or prevention of Alzheimer's disease or to prevent the cytotoxic effects of beta amyloid protein associated with Alzheimer's disease or to treat or prevent fibriologenic diseases, to decrease oxidation in biological samples or to treat or prevent other diseases or conditions in which free radicals or oxidative stress play a role, the substantially optically pure diastereomeric forms of S-adenosyl-L-methionine salts or a non-racemic mixture of (S,S)-S-adenosyl-L-methionine and (R,S)-S-adenosyl-L-methionine and their salts as envisioned by the present invention may be combined with an indole compound such as indole-3-propionic acid (doses may range from 20 mg to 1-2 grams per day), melatonin or the water soluble indole-3-propionic acid derivatives that are the subject of the U.S. patent application Ser. No. 10/631,122 which is incorporated by reference herein in its entirety as well but not limited to folic acid at 50 to 1000 mcg; vitamin B6 at 10 to 2000 mg; vitamin B12 at 10-3000 mcg and biopterin. This composition can, of course, be combined further with conventional Alzheimer's drugs in one convenient capsule or pill form for ease of administration and to increase patient compliance.

In yet another embodiment, the substantially optically pure diastereomeric forms of S-adenosyl-L-methionine salts or a non-racemic mixture of (S,S)-S-adenosyl-L-methionine and (R,S)-S-adenosyl-L-methionine and their salts of this present invention may be combined with other antioxidants such as glutathione, lipoic acid vitamin E, vitamin C, N-acetylcysteine and the like for enhanced synergistic effects as well as for ease of administration and may also include but not limited to folic acid at 50 to 1000 mcg; vitamin B6 at 10 to 2000 mg; vitamin B12 at 10-3000 mcg and biopterin.

In yet a further embodiment the substantially optically pure diastereomeric forms of S-adenosyl-L-methionine salts or a non-racemic mixture of (S,S)-S-adenosyl-L-methionine and (R,S)-S-adenosyl-L-methionine and their salts can be combined with other natural (for example, such as Hypericum or St. John's wort the effective clinical dose of which is known in the literature) as well as synthetic drugs for the treatment of depression. As their modes of action differ, the combination of a methyl donor such as S-adenosyl-L-methionine and any other drug for the treatment of depression such as an SSRI, (doses at currently well known amounts) epinephrine, tryptophan, norepinephrine, or other drugs having a substantially different mechanism of action for the synergistic treatment of depression is envisioned. The composition may also include but is not limited to folic acid at 50 to 1000 mcg; vitamin B6 at 10 to 2000 mg; vitamin B12 at 10-3000 mcg and biopterin.

In yet a further embodiment the substantially optically pure diastereomeric forms of S-adenosyl-L-methionine salts or a non-racemic mixture of (S,S)-S-adenosyl-L-methionine and (R,S)-S-adenosyl-L-methionine and their salts can be combined with folic acid at 50 to 1000 mcg; vitamin B6 at 10 to 2000 mg; vitamin B12 at 10-3000 mcg and biopterin to treat conditions associated with protein, RNA, or DNA hypomethylation (for example, autoimmune diseases, cancers, atherosclerosis).

In a preferred embodiment, substantially optically pure diastereomeric forms of S-adenosyl-L-methionine salts or a non-racemic mixture of (S,S)-S-adenosyl-L-methionine and (R,S)-S-adenosyl-L-methionine and their salts and the additives of this current patent application are administered to a warm-blooded animal as a pharmaceutical, prophylactic or cosmetic composition containing at least one substantially optically pure diastereomeric form of S-adenosyl-L-methionine salt or a non-racemic mixture of (S,S)-S-adenosyl-L-methionine and (R,S)-S-adenosyl-L-methionine and their salts in combination with at least one pharmaceutically, prophylactically or cosmetically acceptable carrier or diluent. Administration may be accomplished by systemic or topical application, with the preferred mode dependent upon the type and location of the conditions to be treated. Frequency of administration may vary, and is typically accomplished by daily administration.

In another embodiment, a pharmaceutical composition of the non-racemic ratio of (S,S)-S-adenosyl-L-methionine to (RS)-S-adenosyl-L-methionine (and their salts) is preferably from about 80.01% to about 100% of (S,S)-S-adenosyl-L-methionine to about 19.99% to about 0.0% by weight of (R,S)-S-adenosyl-L-methionine and the additives of this current patent application are in the doses mentioned above.

In yet another embodiment, a pharmaceutical composition of the non-racemic ratio of (S,S)—S-adenosyl-L-methionine to (R,S)-S-adenosyl-L-methionine (and their salts) is more preferably from about 82% to about 96.009% of (S,S)-S-adenosyl-L-methionine to about 19.009% to about 3.001% by weight of (R,S)-S-adenosyl-L-methionine.

In yet a further embodiment, a pharmaceutical composition of the non-racemic ratio of (S,S)—S-adenosyl-L-methionine to (R,S)-S-adenosyl-L-methionine (and their salts) is most preferably from about 80.001% to about 95% of (S,S)-S-adenosyl-L-methionine to about 19.009% to about 5% by weight of (R,S)-S-adenosyl-L-methionine.

Systemic administration may be achieved, for example, by injection (e.g., intramuscular, intravenous, subcutaneous or intradermal) or oral delivery of the composition to the warm-blooded animal. Suitable carriers and diluents for injection are known to those skilled in the art, and generally are in the form of an aqueous solution containing appropriate buffers and preservatives. Oral delivery is generally accomplished by formulating the composition in a liquid or solid form, such as a tablet or capsule, by known formulation techniques.

Topical administration may be accomplished, for example, by formulating the composition as solution, cream, gel, ointinent, powder, paste, gum or lozenge using techniques known to those skilled in the formulation field. As used herein, topical administration includes delivery of the composition to mucosal tissue of the mouth, nose and throat by, for example, spray or mist application, as well as to the vagina and rectum by, for example, suppository application.

The following examples shows how substantially optically pure diastereomeric forms of S-adenosyl-L-methionine salts or defined non-racemic ratios of (S,S)-S-adenosyl-L-methionine to (R,S)-S-adenosyl-L-methionine and their salts as well as the additives of this present invention may be used clinically. This example is given to illustrate the present invention, but not by way of limitation. Accordingly, the scope of this invention should be determined not by the embodiments illustrated, but rather by the appended claims and their legal equivalents.

### EXAMPLE 1

A composition containing an enteric-coated tablet form optically pure diastereomeric form of S-adenosyl-L-methionine salts or a non-racemic mixture of (S,S)-S-adenosyl-L-methionine and (R,S)-S-adenosyl-L-methionine and their salts (in this specific example, 1,4 butanedisulfonate salt) 400 mg, folic acid at 50 mcg; vitamin B6 at 50 mg; vitamin B12 at 1000 mcg is administered twice daily in an open, non-blind study of 10 volunteers who are depressed and who give informed consent. All patients have normal results on pre-study medical examinations, including laboratory examinations. Patients receive the composition in an enteric-coated tablet form twice daily for 14 days or until remission of depression symptoms. The 10 patients satisfy the DSM-III criteria for a major depressive episode. Patients' symptoms are monitored daily using the Hamilton Rating Scale for Depression.

### EXAMPLE 2

A composition containing an enteric-coated tablet form substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, disulfate tosylate) 400 mg, folic acid at 50 mcg; vitamin B6 at 50 mg; vitamin B12 at 1000 mcg is administered twice daily in an open, non-blind study of 10 volunteers who suffer from Alzheimer's disease who give informed consent. Patients receive the composition in an enteric-coated tablet form twice daily for one year. Clinically acceptable testing for stabilization of Alzheimer's disease is undertaken to determine the effect of the composition on the symptoms of the disease.

### EXAMPLE 3

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, disulfate tosylate) 800 mg, folic acid at 50 mcg; vitamin B6 at 50 mg; vitamin B12 at 1000 mcg is administered twice daily in an open, non-blind study of 10 volunteers who suffer from alcoholic cirrhosis of the liver in early stage (Childs B) who give informed consent. Patients receive the composition in an enteric-coated tablet form twice daily for 180 days. Liver function studies as well as pre and post liver biopsy results can be used to monitor the effect of the composition in the treatment of alcoholic cirrhosis of the liver.

### EXAMPLE 4

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from viral hepatitis of the liver who give informed consent. Patients receive the composition in an enteric-coated tablet form twice daily for 180 days. Liver function studies as well as pre and post liver biopsy results can be used to monitor the effect of the composition in the treatment of viral cirrhosis of the liver.

### EXAMPLE 5

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from viral hepatitis of the liver who give informed consent. Patients receive the composition in an enteric-coated tablet form twice daily for 180 days. Liver function studies as well as pre and post liver biopsy results can be used to monitor the effect of the composition in the prevention of viral cirrhosis of the liver.

### EXAMPLE 6

A composition containing an enteric-coated tablet form of substantially optically pure diastereomeric form of S-adenosyl-L-methionine salts or a non-racemic mixture of (S,S)-S-adenosyl-L-methionine and (RS)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from total parenteral nutrition induced hepatitis of the liver who give informed consent. Patients receive the composition in an enteric-coated tablet form twice daily for 180 days. Liver function studies can be used to monitor the effect of the composition in the prevention of total parenteral nutrition induced hepatitis of the liver.

### EXAMPLE 7

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from total parenteral nutrition induced hepatitis of the liver who give informed consent. Patients receive the composition in an enteric-coated tablet form twice daily for 180 days. Liver function studies can be used to monitor the effect of the composition in the treatment of total parenteral nutrition induced hepatitis of the liver.

### EXAMPLE 8

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from osteoarthritis who give informed consent. Patients receive the composition in an enteric-coated tablet form twice daily for 60 days. Clinically acceptable testing methodologies to assess the efficacy of the composition are well known, for example, as found in the following citation which is incorporated herein in its entirety: BMC Musculoskelet Disord. 2004 Feb. 26;5(1):6.S-adenosyl methionine (SAMe) versus celecoxib for the treatment of osteoarthritis symptoms: a double-blind cross-over trial. Najm W I, Reinsch S, Hoehler F, Tobis J S, Harvey P W.

### EXAMPLE 9

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from rheumatoid arthritis who give informed consent. Patients receive the composition in an enteric-coated tablet form twice daily for 60 days. Clinically acceptable testing methodologies to assess the efficacy of the composition are well known, for example, as found in the following citation which is incorporated herein in its entirety: BMC Musculoskelet Disord. 2004 Feb. 26;5(1):6.S-adenosyl methionine (SAMe) versus celecoxib for the treatment of osteoarthritis symptoms: a double-blind cross-over trial. Najm W I, Reinsch S, Hoehler F, Tobis J S, Harvey P W.

### EXAMPLE 10

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from global DNA hypomethylation who give informed consent. Patients receive the composition in an enteric-coated tablet form twice daily for 60 days. Clinically acceptable testing methodologies to assess the efficacy of the composition are well known, for example, as found in the following citation which is incorporated herein in its entirety: Mol Carcinog. 2004 February;39(2):79-84. Modulation of DNA hypomethylation as a surrogate endpoint biomarker for chemoprevention of colon cancer. Tao L, Wang W, Kramer P M, Lubet R A, Steele V E, Pereira M A.

### EXAMPLE 11

A composition containing an IV form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from global. DNA hypomethylation who give informed consent. Patients receive the composition in an IV infusion form twice daily for 60 days. Clinically acceptable testing methodologies to assess the efficacy of the composition are well known, for example, as found in the following citation which is incorporated herein in its entirety: Mol Carcinog. 2004 February;39(2):79-84. Modulation of DNA hypomethylation as a surrogate endpoint biomarker for chemoprevention of colon cancer. Tao L, Wang W, Kramer P M, Lubet R A, Steele V E, Pereira M A.

### EXAMPLE 12

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from breast cancer who give informed consent. Patients receive the composition twice daily for three years. Clinically acceptable testing methodologies to assess the efficacy of the composition to prevent breast cancer metastasis are well known.

### EXAMPLE 13

A composition containing an enteric-coated tablet form of substantially optically pure diastereomeric form of S-adenosyl-L-methionine salts or a non-racemic mixture of (S,S)-S-adenosyl-L-methionine and (R,S)-S-adenosyl-L-methionine and their salts (in this specific case, 1,4 butane disulfonate) 800 mg, folic acid at 50 mcg; vitamin B6 at 50 mg; vitamin B12 at 1000 mcg is administered twice daily in an open, non-blind study of 10 volunteers who suffer from mild cognitive impairment who give informed consent. Patients receive the composition twice daily for 180 days. Clinically acceptable testing methodologies to assess the efficacy of the composition to treat mild cognitive impairment are well known, for example, as found in the following citation which is incorporated herein in its entirety: J Intern Med 2004 September;256(3):183-94. Mild cognitive impairment as a diagnostic entity. Petersen R C.

I claim:

1. A composition useful for the treatment of conditions associated with RNA or DNA genome hypomethylation comprising substantially optically pure (S,S)-S-adenosyl-L-methionine or a defined non-racemic ratio of (S,S)S-adenosyl-L-methionine to (RS) S-adenosyl-L-methionine and their pharmaceutically acceptable salts in combination with additives that enhance the activity of the substantially optically pure (S,S)S-adenosyl-L-methionine or a defined non-racemic ratio of (S,S)S-adenosyl-L-methionine to (R,S)S-adenosyl-L-methionine and their pharmaceutically acceptable salts.

2. The composition of claim 1 in which the defined non-racemic ratio of (S,S)S-adenosyl-L-methionine salt:(R,S)S-adenosyl-L-methionine salt is from about 82% to 100%: 18% to 0% by weight.

3. The composition of claim 1 in which the additives that enhance the activity of the substantially optically pure (S,S)-S-adenosyl-L-methionine or a defined non-racemic ratio of (S,S)S-adenosyl-L-methionine to (R,S)S-adenosyl-L-methionine and their pharmaceutically acceptable salts are chosen from the group consisting of: vitamin B6, folic acid, St. John's Wort, vitamin B12, melatonin, indole-3-propionic acid, vitamin C, vitamin E, tryptophan, selective seratonin reuptake inhibitors, COX 2 inhibitors, anticancer drugs, acetylcholinesterase inhibitors, gama secretase inhibitors, or gaba inhibitors or any mixtures thereof.
